(19) United States
(12) Patent Application Publication
(10) Pub. No.: US 2007/0219379 A1
(43) Pub. Date: Sep. 20, 2007

(54) Ionic Liquid and Process for Producing the Same

(75) Inventors: Toshiyuki Itoh, Tottori-shi (JP); Hiroyuki Furutani, Takasuki-shi (JP); Masamitsu Tachibana, Osaka (JP); Yasuhiro Tsukada, Koube-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Kaneka Corporation, Osaka-shi (JP)

(21) Appl. No.: 11/578,306

(22) PCT Filed: Apr. 26, 2005
(86) PCT No.: PCT/JP05/07916

§ 371 Date: Oct. 13, 2006

(30) Foreign Application Priority Data
Apr. 28, 2004 (JP) 2004-133168
Apr. 30, 2004 (JP) 2004-136301

(51) Int. Cl. C07D 233/58; H01M 10/40; H01M 6/16

(52) U.S. Cl. 548/343.1; 568/74

(57) ABSTRACT

The present invention provides an ionic liquid that can widely be used as materials of various electrical devices and solvents for various reactions and that exhibits hydrophobicity causing phase separation from water, and a process for producing the ionic liquid. The ionic liquid contains at least one anion selected from the group consisting of a fluoroalkylsulfate anion, a fluorocycloalkylsulfate anion, and a fluorobenzylsulfate anion and exhibits hydrophobicity causing phase separation from water. The halide ion concentration of the ionic liquid may be 70 ppm or less.

### TECHNICAL FIELD

The present invention relates to an ionic liquid that exhibits hydrophobicity causing phase separation from water and a process for producing the ionic liquid. The present invention particularly relates to an ionic liquid that has a low halide ion concentration and that can be suitably applied to materials for various electrical devices, solvents for various reactions, etc., and a process for producing the ionic liquid.

### BACKGROUND ART

In general, an ionic liquid is composed of a combination of a cation, such as imidazolium, and an appropriate anion (Br⁻, Cl⁻, RSO₄⁻, BF₄⁻, PF₆⁻, (CF₃SO₂)₂N⁻, or the like). Since the ionic liquid exhibits high ionic conductivity and excellent thermal stability, applications to electrolytes for batteries and capacitors, solvents for various chemical reactions, etc., have been widely studied.

In general, an ionic liquid is hydrophilic except for those containing particular expensive anions such as PF₆⁻, (CF₃SO₂)₂N⁻, or the like. For example, an ionic liquid containing a 1-butyl-3-methylimidazolium cation and BF₄⁻ is hydrophilic. Such hydrophilic ionic liquids are highly hygroscopic and thus their usage is limited.

The most common process for producing the ionic liquid is an ion exchange reaction in which a halide containing a cation for a target ionic liquid is reacted with a salt containing an anion for the target ionic liquid (for example, refer to nonpatent document 1, p. 4). However, this process disadvantageously causes halide ions produced during the reaction to remain in the ionic liquid.

Since the ionic liquid is not vaporized even at high temperature, the ionic liquid cannot be purified by distillation. Removal of impurities, such as halide ions, from an ionic liquid is not easy once the liquid is synthesized (for example, refer to nonpatent document 1, p. 25). Accordingly, concentrations of halide ions contained as impurities frequently differ from one ionic liquid to another even when the ionic liquids are of the same type, and this has resulted in differences in physical properties such as melting point and density. Evidently, the quality of the ionic liquid has not been sufficiently guaranteed.

For example, for 1-ethyl-3-methylimidazoliumhexafluorophosphate, different values of melting point, namely, −16.15° C. and −17.15° C., and different values of density, namely, 1.52 g/cm³ (21.85° C.) and 1.518 g/cm³, have been reported (refer to nonpatent documents 2 to 5).

There is also a report that the ionic liquid can be used as a solvent for synthetic organic reaction. However, in order to carry out, for example, enzymatic reaction in an ionic liquid, the concentration of the impurities, namely, halide ions, poses a problem. When an ionic liquid is used as a solvent for enzymatic reaction, halide ions as impurities contained in the solvent deactivate the enzyme, and the reaction would not proceed sufficiently. Recently, a report has been made on enzymatic reaction using an ionic liquid as a reaction solvent; however, since enzymatic activity is greatly affected by trace amounts of impurities, different results have been reported from the same enzyme (for example, refer to nonpatent document 6).
[Nonpatent document 1] “Ionic Liquids: The Front and Future of Material Development” edited by Hiroyuki OHNO, CMC Publishing Co., Ltd. (2003)
[Nonpatent document 2] P. Bonhote et al., Inorg. Chem., 35, p. 1168 (1996)
[Nonpatent document 3] A. Noda et al., J. Phys. Chem. B, 105, p. 4603 (2001)
[Nonpatent document 4] A. B. McEwen et al., Journal of The Electrochemical Society, 146, p. 1687 (1999)
[Nonpatent document 5] H. L. Ngo et al., Thermochim. Acta, 97, p. 357 (2000)
[Nonpatent document 6] R. A. Sheldon et al., Green Chem. 4, p. 147 (2002)

### DISCLOSURE OF INVENTION

Problems to be Solved by the Invention

In view of the above-described circumstances, an object of the present invention is to provide an ionic liquid that can be widely used as materials of various electrical devices and solvents for various reactions and that shows hydrophobicity causing phase separation from water, and a process for producing such an ionic liquid.

Means for Solving the Problems

The present invention provides an ionic liquid that contains at least one anion selected from the group consisting of a fluoroalkylsulfate anion, a fluorocycloalkylsulfate anion, and a fluorobenzylsulfate anion and that exhibits hydrophobicity causing phase separation from water.

The ionic liquid of the present invention may contain at least one cation selected from the group consisting of a cation having a heterocyclic skeleton containing at least one nitrogen atom and an ammonium cation. The concentration of halide ions can be adjusted to 70 ppm or less or even 45 ppm or less.

The present invention also provides a process for producing an ionic liquid, comprising:

a synthetic step of reacting at least one ammonium salt selected from the group consisting of ammonium fluoroalkylsulfate, ammonium fluorocycloalkylsulfate, and ammonium fluorobenzylsulfate with at least one halide selected from the group consisting of a halide having a heterocyclic skeleton containing at least one nitrogen atom and an ammonium halide, and removing the resulting ammonium halide to prepare an ionic liquid; and

a purification step of bringing the ionic liquid into contact with alumina powder to remove halide ions contained in the ionic liquid.

In the purification step of the process for producing the ionic liquid of the present invention, column chromatography with a column packed with alumina powder may be employed to remove the halide ions contained in the ionic liquid. The alumina powder may be neutral alumina powder.

### ADVANTAGES OF THE INVENTION

As described above, the present invention can provide an ionic liquid that exhibits hydrophobicity causing phase separation from water and a process for producing the ionic liquid.

### BRIEF DESCRIPTION OF THE DRAWINGS

FIG. 1 is a photograph showing the hydrophobicity and hydrophilicity of an ionic liquid. Part (a) shows how a hydrophobic ionic liquid undergoes phase separation from water, and part (b) shows how a hydrophilic ionic liquid mixes with water by forming a single phase.

FIG. 2 is a schematic diagram showing a reaction scheme of enzymatic reaction.

### BEST MODE FOR CARRYING OUT THE INVENTION

<Hydrophobicity that Causes Phase Separation from Water>

The ionic liquid of the present invention contains at least one anion selected from the group consisting of a fluoroalkylsulfate anion, a fluorocycloalkylsulfate anion, and a fluorobenzylsulfate anion as the anion of the ionic liquid and exhibits hydrophobicity that causes phase separation from water. When at least one of the anions described above is contained as the anion of the ionic liquid, hydrophobicity that causes phase separation from water can be attained. For the purpose of this specification, “liquid that exhibits hydrophobicity that causes phase separation from water” is defined as a liquid that undergoes phase separation from water by forming two phases when the liquid is mixed with deionized water and left to stand for 12 hours at room temperature (25° C.). Furthermore, “liquid that exhibits hydrophilicity” is defined as a liquid that remains mixed without phase separation when mixed with deionized water and left to stand for 12 hours at room temperature (25° C.).

<Fluoroalkylsulfate Anion>

The fluoroalkylsulfate anion is not particularly limited. However, from the standpoint of enhancing the hydrophobicity, the number of carbon atoms in the fluoroalkyl group is preferably 4 or more, and the number of fluorine atoms in the fluoroalkyl group is preferably 4 or more. Examples of the fluoroalkylsulfate anion include a 4,4,5,5,5-pentafluoro-1-pentanesulfate anion, a 2,2,3,3,4,4,4-heptafluoro-1-butylsulfate anion, a 2,2,3,3,4,4,5,5-octafluoropentanesulfate anion, and a 1H,1H-pentadecafluoro-1-octanesulfate anion.

<Fluorocycloalkylsulfate Anion>

The fluorocycloalkylsulfate anion is not particularly limited. However, from the standpoint of enhancing the hydrophobicity, the number of carbon atoms in the fluorocycloalkyl group is preferably 4 or more, and the number of fluorine atoms in the fluorocycloalkyl group is preferably 4 or more. An example of the fluorocycloalkylsulfate anion is a 2,2,3,3,4,4,5,5,6,6-decafluorocyclohexylsulfate anion.

<Fluorobenzylsulfate Anion>

The fluorobenzylsulfate anion is not particularly limited. However, from the standpoint of enhancing the hydrophobicity, the number of fluorine atoms in the fluorobenzyl group is preferably 3 or more. An example of the fluorobenzylsulfate anion is a 2,3,4,5,6-pentafluorobenzylsulfate anion.

<Cation Constituting the Ionic Liquid>

The ionic liquid of the present invention preferably contains at least one cation selected from the group consisting of a cation having a heterocyclic skeleton having one or more nitrogen atoms and an ammonium cation. When at least one of these cations is contained and combined with the anion described above, an ionic liquid having high ionic conductivity and thermal stability is obtained.

The cation having a heterocyclic skeleton containing one or more nitrogen atoms is not particularly limited. However, from the standpoint of attaining high ionic conductivity and thermal stability, an imidazolium cation represented by chemical formula (1), a pyridinium cation represented by chemical formula (2), a pyrrolidinium cation represented by chemical formula (3), a triazine derivative cation represented by chemical formula (4), and the like are preferred. In each of chemical formulae (1) to (4), R₁ to R₁₉ each independently represent H, an alkyl group, an alkenyl group, or an alkoxy group.

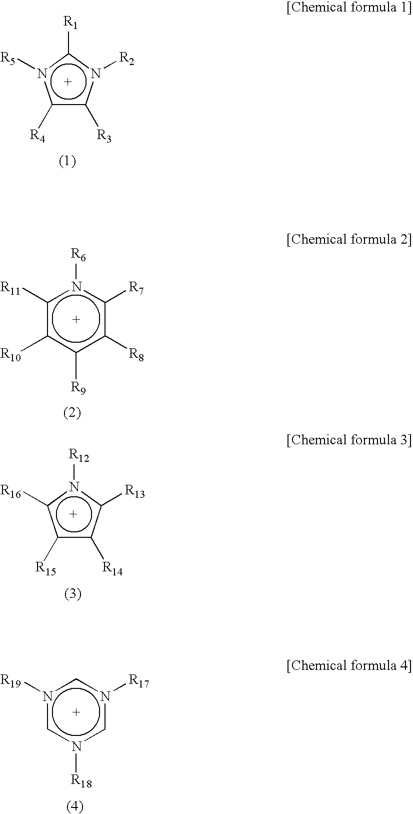

The ammonium cation is not particularly limited. An example thereof is an ammonium cation represented by chemical formula (5). In chemical formula (5), R₂₀ to R₂₃ each independently represent H, an alkyl group, an alkenyl group, or an alkoxy group.

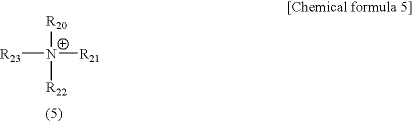

Examples of the imidazolium cation include a 1-ethylimidazolium cation, a 1,3-diethylimidazolium cation, a 1-ethyl-3-methylimidazolium cation, a 1-butyl-3-methylimidazolium cation, a 1-isobutyl-3-methylimidazolium cation, a 1-ethyl-2,3-dimethylimidazolium cation, a 1-butyl-2,3-dimethylimidazolium cation, and a 1-(2,2,2-trifluoroethyl)-3-methylimidazolium cation.

Examples of the pyridinium cation include a pyridinium cation, an N-ethylpyridinium cation, and an N-butylpyridinium cation.

Examples of the pyrrolidinium cation include a pyrrolidinium cation, a 2-methylpyrrolidinium cation, a 3-ethylpyrrolidinium cation, a 3-butylpyrrolidinium cation, and a 2-butylpyrrolidinium cation.

Examples of the triazine derivative cation include a 1,3-diethyl-5-methyltriazinium cation, a 1,3-dimethyl-5-ethyltriaziniuim cation, a 1,3-diethyl-5-butyltriazinium cation, a 1,3-dibutyl-5-methyltriazinium cation, and a 1,3,5-tributyltriazinium cation=bis((trifluoromethyl)sulfonyl)amide.

Examples of the ammonium cation include a tetramethylammonium cation, a trimethylethylammonium cation, a trimethylbutylammonium cation, a trimethyloctylammonium cation, a triethylbutylammonium cation, and a tetrabutylammonium cation.

<Step of Synthesizing Ionic Liquid>

The ionic liquid of the present invention, which contains at least one anion selected from the group consisting of a fluoroalkylsulfate anion, a fluorocycloalkylsulfate anion, and a fluorobenzylsulfate anion as an anion of ionic liquid and at least one cation selected from the group consisting of a cation having a heterocyclic skeleton containing one or more nitrogen atoms and an ammonium cation as a cation of the ionic liquid is produced as follows.

First, at least one ammonium salt [NH₄⁺][RF—OSO₃⁻] selected from the group consisting of ammonium fluoroalkylsulfate, ammonium fluorocycloalkylsulfate, and ammonium fluorobenzylsulfate is reacted with at least one halide [R(N)⁺][X⁻] selected from the group consisting of a halide having a heterocyclic skeleton containing one or more nitrogen atoms and an ammonium halide, thereby causing ion exchange reaction shown in chemical reaction formula (6) below and giving ammonium halide ([NH₄⁺][X⁻]) and [R(N)⁺][RF—OSO₃⁻]. The ammonium halide is obtained as a deposit. Next, the deposited ammonium halide is removed to obtain an ionic liquid composed of [R(N)⁺][RF—OSO₃⁻]. This is the step of synthesizing the ionic liquid.

[Chemical Formula 6]

[NH₄⁺][RF—OSO₃⁻]+[R(N)⁺][X⁻]→[NH₄⁺][X]+[R(N)⁺][RF—OSO₃⁻] (6)

<Step of Purifying Ionic Liquid>

The above-described ionic liquid contains residual halide ions ([X⁻]) which has not been deposited as ammonium halide. Thus, the ionic liquid is brought into contact with alumina powder to remove the halide ions. This is the step for purifying the ionic liquid.

In the purification process of the ionic liquid described above, the method of bringing the ionic liquid into contact with the alumina powder is not particularly limited. The ionic liquid may be mixed with a solvent and then alumina powder may be added to the resulting solution, followed by stirring. Alternatively, the ionic liquid may be mixed with a solvent and then passed through a column packed with alumina powder. From the standpoint of high purification efficiency and working efficiency, a column chromatographic process using a column packed with alumina powder is preferable.

From the standpoint of purification efficiency, the alumina powder is preferably neutral alumina powder.

<Concentration of Halide Ions>

As a result of the above-described production process including the purification step, the halide ion concentration in the ionic liquid can be decreased to 70 ppm or less or even 45 ppm or less. An ionic liquid with a halide ion concentration of 70 ppm or less causes a smaller decrease in enzymatic activity during the enzymatic reaction and is thus preferable as an enzymatic reaction solvent. An ionic liquid with a halide ion concentration of 45 ppm or less causes a further smaller decrease in enzymatic activity during the enzymatic reaction and is thus more preferable as an enzymatic reaction solvent.

<Ionic Liquid with 95% or Higher Liquid-Chromatographic Purity>

We also disclose the invention described below.

An ionic liquid having a liquid-chromatographic purity of 95% or more and/or a process for producing the ionic liquid provides an ionic liquid that has constant quality and can be used as, for example, a solvent for enzymatic reaction and/or a process for producing such an ionic liquid.

The ionic liquid with a liquid-chromatographic purity of 95% or more can also be obtained through a purification step by column chromatography or a purification step of passing the liquid through alumina powder. Any desired ionic liquid can be processed into an ionic liquid with a liquid-chromatographic purity of 95% or more through the purification step described below.

The present invention also discloses an ionic liquid having a liquid-chromatographic purity of 95% or more.

The present invention also discloses an ionic liquid having a purity of 95% or more and a halide ion concentration of 70 ppm or less.

The present invention also discloses an ionic liquid having a liquid-chromatographic purity of 95% or more, the purity being determined by reverse phase liquid chromatography.

The present invention also discloses a process for producing an ionic liquid having a liquid-chromatographic purity of 95% or more, the process including a purification step of removing by column chromatography impurities and/or halide ions produced during the production.

The present invention also discloses a process for producing an ionic acid having a liquid-chromatographic purity of 95% or more and a halide ion concentration of 70 ppm or less, the process including a purification step of removing by column chromatography impurities and/or halide ions produced during the production.

The present invention also discloses a process for producing an ionic liquid having a liquid-chromatographic purity of 95% or more, the process including a purification step of passing an ionic liquid containing impurities and/or halide ions produced during the production process through alumina powder.

The present invention also discloses a process for producing an ionic liquid having a liquid-chromatographic purity of 95% or more and a halide ion concentration of 70 ppm or less, the process including a purification step of passing an ionic liquid containing impurities and/or halide ions produced during the production through alumina powder.

The present invention also discloses a process for producing an ionic liquid by using neutral activated alumina as the alumina powder.

The present invention relates to a high-purity ionic liquid and a production process therefor. In particular, it relates to a high-quality, excellent ionic liquid that has low concentrations of impurities and/or halide ions produced during the production, that is suitable as a solvent for enzymatic reaction, and/or that can be applied to electrical devices.

The background art is the same as those described above. A process of regenerating an ionic liquid repeatedly used has not been established to date.

According to the present invention, free halogen species generated during the reaction and/or impurities produced in the reaction system can be remarkably easily removed. Thus, it may be possible to provide an ionic liquid that can be applied to nonaqueous batteries, electrochemical capacitors, electroplating, and the like in which halogen or contaminants would degrade characteristics, at industrially advantageous costs.

The objects to be achieved by the present invention are as follows.

An object of the present invention is to provide an ionic liquid of constant quality and usable as, e.g., a solvent for enzymatic reaction, and a process for producing such an ionic liquid.

The object can be achieved by the following means.

That is, the present invention provides an ionic liquid having a molten salt purity of 95% or more determined by liquid chromatography and a halide ion concentration of 70 ppm or less. This room-temperature molten salt constantly exhibits particular quality and can yield a reaction rate of a particular value or more when used as a solvent for enzymatic reaction, for example. Since the purity is high, the conductivity is maintained at a particular level. Thus, there is a possibility that the ionic liquid can be applied to batteries, electrochemical capacitors, electroplating, and the like at industrially advantageous costs.

The production process of the present invention is a process for producing an ionic liquid, the process including a purification step of removing halide ions by column chromatography.

The production process of the present invention is also a process for producing an ionic liquid, including a purification step of passing an ionic liquid containing halide ions as impurities through alumina powder.

As the alumina powder used in this purification step, neutral activated alumina is preferred.

The most preferable purification step uses column chromatography with a neutral activated alumina column packing since a high-purity ionic liquid can be efficiently produced.

The advantages of the invention are as follows.

The ionic liquid of the present invention exhibits constant quality, can be used as a solvent for, e.g., enzymatic reaction, and can be produced easily and efficiently.

The embodiments of the present invention are as follows.

The ionic liquid is mixed with acetone. The resulting acetone solution is poured into a cylindrical dropping funnel packed with neutral activated alumina (type I) so as to remove acetone from the acetone solution. As a result, a room-temperature molten salt substantially free (50 ppm or less) of impurities such as impurities produced during the reaction and halide ions can be obtained. Examples of the cation in the ionic liquid to which this purification method can be suitably applied include an imidazolium cation (formula (1)), a pyridinium cation (formula (2)), an ammonium cation (formula (5)), and a triazine derivative cation (formula (4)).

Examples of the anion in the ionic liquid to which this purification method can be suitably applied include AlCl₄⁻, BF₄⁻, PF₆⁻, NO₃⁻, RCOO⁻, RSO₃⁻, NH₂CHRCOO⁻, and SO₄²⁻ (wherein R represents H, an alkyl group, or an alkyloxy group).

The ionic liquid of the present invention exhibits constant quality and can yield a reaction rate of a particular value or more when used as a solvent for enzymatic reaction, for example. In particular, an ionic liquid having a purity of 95% or more can remarkably increase the rate of enzymatic reaction and is thus preferable. The purity is preferably 95% or more and more preferably 98% or more.

The ionic liquid exhibits constant quality and can yield a reaction rate of a particular value or more when used as a solvent for enzymatic reaction, for example. In particular, an ionic liquid having a free halide ion concentration of 70 ppm or less can remarkably increase the rate of enzymatic reaction and is thus preferred. The concentration is preferably 70 ppm or less and more preferably 50 ppm or less.

The invention related to the ionic liquid having a liquid-chromatographic purity of 95% or more can be summarized as follows.

An object is to provide an ionic liquid that exhibits constant quality and that can be used as a solvent for, e.g., enzymatic reaction and a process for producing such an ionic liquid.

The means for achieving the object are as follows. In particular, the object can be achieved by an ionic liquid having a liquid-chromatographic purity of 95% or more and a halide ion concentration of 70 ppm or less, the ionic liquid being produced by:
(1) removing halide ion impurities in the ionic liquid using activated alumina;
(2) removing halide ion impurities in the ionic liquid using neutral activated alumina among activated alumina; and
(3) efficiently removing impurities produced during the reaction and/or halide ion impurities from the ionic liquid using column chromatography with a column packed with neutral activated alumina.

Although EXAMPLES are described below, the present invention is not limited by these examples.

### EXAMPLES

### Example 1

(1) Synthesis of Ionic Liquid

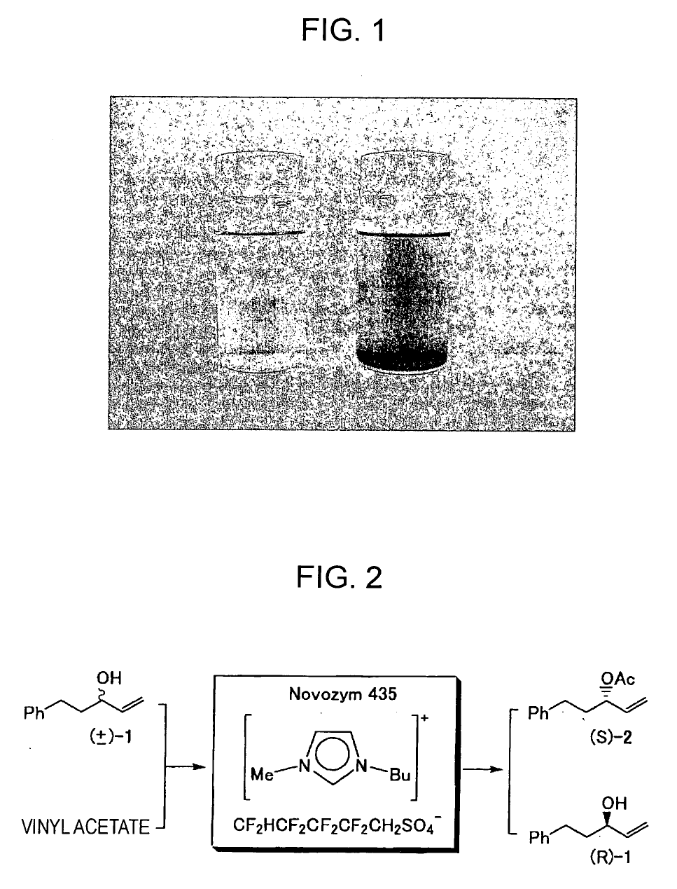

To a thoroughly dried 500 cm³ separable flask, a mixing impeller and a Liebig reflux condenser were mounted and 35.1 g (0.18 mol) of 3-butyl-1-methylimidazolium chloride and 20 cm³ of DMF were added, and the resulting mixture was thoroughly stirred. Then, 59.1 g (0.18 mol) of ammonium 2,2,3,3,4,4,5,5-octafluoropentanesulfate and 100 cm³ of acetone were rapidly added to the separable flask. Upon completion of the addition, the mixture was stirred at room temperature (25° C.) for 12 hours. Deposited ammonium chloride was removed on celite, and the acetone in the recovered acetone solution was distilled off under a reduced pressure on an evaporator. The residue was washed and concentrated with an n-hexane/ethyl acetate (volume ratio: 3/1) mixed solvent, again mixed with acetone, and decolorized with activated carbon. From the again-recovered acetone solution, acetone was distilled off under a reduced pressure on an evaporator to recover 1-butyl-3-methylimidazolium 2,2,3,3,4,4,5,5-octafluoropentanesulfate as 80.6 g (yield: 95.2%) of a light-brown ionic solution. At room temperature (25° C.), 10 cm³ of the ionic liquid obtained was added to 10 cm³ of ion-exchange water placed in a 30 cm³ sample bottle. The bottle was capped, shaken for 5 minutes at a shaking pitch of 30 cm and 60 reciprocating motions per minute, and left to stand for 10 minutes. As a result, the solution was completely separated into two phases, i.e., an aqueous phase (upper side) and an ionic liquid phase (lower side) as shown in FIG. 1(a), and the ionic liquid exhibited hydrophobicity.

(2) Purification of Ionic Liquid

Next, 5 cm³ of the ionic liquid above was mixed with 10 cm³ of acetone, and the resulting acetone solution was poured into a cylindrical dropping funnel (20 cm³) in which neutral activated alumina (type I) is packed for about 5 cm. The ionic liquid was passed through the funnel under pressurization with an air pump with acetone as a washing solution and then further washed with acetone three times. The resulting acetone solution was concentrated on an evaporator, and the solvent was completely removed by reducing the pressure to 266 Pa (2 torr). The halide ion concentration in the ionic liquid thus purified measured by ion chromatography (analytic system: DX-500 (GP40 and ED40) produced by Dionex, column: IonPac AG12A, and AS12A (4 mm (dia)×250 mm (length)), eluent: NaHCO₃ (0.3 mM)+Na₂CO₃ (2.7 mM), eluent flow rate: 1.2 cm³/min, sample injection: 25×10⁻³ cm³, detector: conductometric detection) was 43 ppm.

As shown in EXAMPLES 7 to 9 below, the ionic liquid which was not purified with neutral activated alumina had a halide ion concentration of 88 ppm or more. In other words, neutral activated alumina is extremely effective for removing halide ions in the ionic liquid.

(3) Evaluation of Properties of Ionic Liquid

The ionic conductivity of the purified ionic liquid measured by an impedance method (analytic system: electrochemical analyzer ALS 608B produced by ALS) was satisfactory, i.e., 3×10⁻³ S/cm.

Furthermore, 1.5 cm³ of the purified ionic liquid was sampled and mixed with 25 cm³ of Novozym 435 (lipase enzyme immobilized on porous acrylic resin, produced by Novo Nordisk), racemic 5-phenyl-1-penten-3-ol ((±)-1) (49.0 mg, 0.30 mmol), and vinyl acetate (39.0 mg, 0.45 mmol). The resulting mixture was stirred at 35° C. After two hours, 2 cm³ of diethyl ether was added to separate the liquid into two phases, and the procedure of isolating the ether phase with a pipette was conducted 20 times. The collected ether phase was concentrated under vacuum and purified by thin-layer chromatography on silica gel to isolate a product, i.e., acetate (S)-2, and unreacted 5-phenyl-1-penten-3-ol (R)-1. The reaction scheme therefor is shown in FIG. 2. The optical purities (% ee) of the resulting acetate (S)-2 and 5-phenyl-1-penten-3-ol (R)-1 were measured, and conversions c (% conv) were calculated from the results. The conversion c (% conv) calculated was divided by the reaction time (hr) to define the relative rate, and the enzymatic activity was evaluated based on the value of the relative rate. In this example, the relative rate was as high as 21% conv/hr.

As described below, an ionic liquid not purified with neutral activated alumina has a high halide ion impurity content. Moreover, the relative rate (21% conv/hr) obtained here is higher than that of the examples in which enzymatic reaction by Novozym 435 is conducted in an ionic liquid not purified by neutral activated alumina. In other words, purification with neutral activated alumina is extremely effective for removing halide ions in the ionic liquid. The results are shown in Table 1.

### Example 2

An ionic liquid, 1-butyl-3-methylimidazolium 2,2,3,3,4,4,4-heptafluoro-1-butylsulfate was synthesized as in EXAMPLE 1 but with ammonium 2,2,3,3,4,4,4-heptafluoro-1-butylsulfate instead of ammonium 2,2,3,3,4,4,5,5-octafluoropentanesulfate. The resulting ionic liquid was hydrophobic, the halide ion concentration after purification was 44 ppm, and the ionic conductivity was 2.5×10⁻³ S/cm. The same enzymatic reaction as in EXAMPLE 1 was conducted with the purified ionic liquid. The relative rate was as high as 22 %conv/hr. The results are shown in Table 1.

### Example 3

An ionic liquid, 1-butyl-3-methylimidazolium 4,4,5,5,5-pentafluoro-1-pentanesulfate was synthesized as in EXAMPLE 1 but with ammonium 4,4,5,5,5-pentafluoro-1-pentanesulfate instead of ammonium 2,2,3,3,4,4,5,5-octafluoropentanesulfate. The resulting ionic liquid was hydrophobic, the halide ion concentration after purification was 66 ppm, and the ionic conductivity was 3.1×10⁻³ S/cm. The same enzymatic reaction as in EXAMPLE 1 was conducted using the purified ionic liquid. The relative rate was as high as 16% conv/hr. The results are shown in Table 1.

### Example 4

1-Butyl-3-methylimidazolium 1H,1H-pentadecafluoro-1-octanesulfate was synthesized as in EXAMPLE 1 but with ammonium 1H,1H-pentadecafluoro-1-octanesulfate instead of ammonium 2,2,3,3,4,4,5,5-octafluoropentanesulfate. The resulting ionic liquid was hydrophobic, the halide ion concentration after purification was 68 ppm, and the ionic conductivity was 2.4×10⁻³ S/cm. The same enzymatic reaction as in EXAMPLE 1 was conducted with the purified ionic liquid. The relative rate was as high as 17% conv/hr. The results are shown in Table 1.

### Example 5

1-Butyl-3-methylimidazolium 2,3,4,5,6-pentafluorobenzylsulfate was synthesized as in EXAMPLE 1 but with ammonium 2,3,4,5,6-pentafluorobenzylsulfate instead of ammonium 2,2,3,3,4,4,5,5-octafluoropentanesulfate. The resulting ionic liquid was hydrophobic, the halide ion concentration after purification was 51 ppm, and the ionic conductivity was 2.9×10⁻³ S/cm. The same enzymatic reaction as in EXAMPLE 1 was conducted with the purified ionic liquid. The relative rate was as high as 19% conv/hr. The results are shown in Table 1.

### Example 6

1-Butyl-2,3-dimethylimidazolium 2,2,3,3,4,4,4-heptafluoro-1-butylsulfate was synthesized as in EXAMPLE 2 but with 1-butyl-2,3-dimethylimidazolium chloride instead of 1-butyl-3-methylimidazolium chloride. The resulting ionic liquid was hydrophobic, the halide ion concentration after purification was 40 ppm, and the ionic conductivity was 2.3×10⁻³ S/cm. The same enzymatic reaction as in EXAMPLE 1 was conducted with the purified ionic liquid. The relative rate was as high as 25% conv/hr. The results are shown in Table 1.

TABLE 1

| | | | | | | Enzymatic |
|---|---|---|---|---|---|---|
| | | | | | | reaction |
| | | | | Halide | Ionic | relative |
| | | Purification/ | Hydrophilic/ | ion | conductivity | rate |
| | Ionic liquid | washing | hydrophobic | (ppm) | (S/cm) | (% conv/hr) |
| EXAMPLE | 1-Butyl-3- | Neutral | Hydrophobic | 43 | 3.0 × 10⁻³ | 21 |
| 1 | methylimidazolium=2,2,3,3,4,4,5,5- | activated | | | | |
| | octafluoropentanesulfate | alumina | | | | |
| EXAMPLE | 1-Butyl-3- | Neutral | Hydrophobic | 44 | 2.5 × 10⁻³ | 22 |
| 2 | methylimidazolium=2,2,3,3,4,4,4- | activated | | | | |
| | heptafluoro-1-butylsulfate | alumina | | | | |
| EXAMPLE | 1-Butyl-3- | Neutral | Hydrophobic | 66 | 3.1 × 10⁻³ | 16 |
| 3 | methylimidazolium=4,4,5,5,5- | activated | | | | |
| | pentafluoro-1-pentanesulfate | alumina | | | | |
| EXAMPLE | 1-Butyl-3- | Neutral | Hydrophobic | 68 | 2.4 × 10⁻³ | 17 |
| 4 | methylimidazolium=1H,1H- | activated | | | | |
| | pentadecafluoro-1-octanesulfate | alumina | | | | |
| EXAMPLE | 1-Butyl-3- | Neutral | Hydrophobic | 51 | 2.9 × 10⁻³ | 19 |
| 5 | methylimidazolium=2,3,4,5,6- | activated | | | | |
| | pentafluorobenzylsulfate | alumina | | | | |
| EXAMPLE | 1-Butyl-2,3- | Neutral | Hydrophobic | 40 | 2.3 × 10⁻³ | 25 |
| 6 | dimethylimidazolium=2,2,3,3,4,4,4- | activated | | | | |
| | heptafluoro-1-butylsulfate | alumina | | | | |

### Example 7

In EXAMPLE 1, washing with H₂0 was conducted instead of purification of the ionic liquid with neutral activated alumina (type I). The halide ion concentration after washing was 124 ppm, and the ionic conductivity was 0.9×10⁻³ S/cm. The same enzymatic reaction as in EXAMPLE 1 was conducted using the washed ionic liquid. The relative rate was as low as 4.2% conv/hr. Although the causal relationship between the halide ions and the enzymatic activity was not completely identified, the ionic liquid was not sufficiently purified for causing the enzymatic reaction. The results are shown in Table 2.

### Example 8

In EXAMPLE 1, the ionic liquid was washed with a saturated aqueous sodium hydrogen carbonate (room temperature (25° C.)) and H₂O instead of purification with neutral activated alumina (type I). The halide ion concentration after the washing was 98 ppm, and the ionic conductivity was 2.1×10⁻³ S/cm. The same enzymatic reaction as in EXAMPLE 1 was conducted using the washed ionic liquid. The relative rate was as low as 4.2% conv/hr. The results are shown in Table 2.

### Example 9

In EXAMPLE 1, the ionic liquid was purified with MgO/SiO₂ (Florisil produced by Floridin) instead of neutral activated alumina (type I). The halide ion concentration after the purification was 88 ppm, and the ionic conductivity was 2.2×10⁻³ S/cm. The same enzymatic reaction as in EXAMPLE 1 was conducted using the purified ionic liquid. The relative rate was as low as 5.5% conv/hr. The results are shown in Table 2.

### Example 10

In EXAMPLE 1, the ionic liquid was purified over silica gel (Wakogel produced by Wako Pure Chemical Industries, Ltd.) instead of neutral activated alumina (type I). The halide ion concentration after the purification was 98 ppm, and the ionic conductivity was 2.0×10⁻³ S/cm. The same enzymatic reaction as in EXAMPLE 1 was conducted using the purified ionic liquid. The relative rate was as low as 3.1% conv/hr. The results are shown in Table 2.

### Example 11

An ionic liquid (5 mL), namely, 1-butyl-3-methylimidazolium 2,2,3,3,4,4,5,5-octafluoropentane sulfate composed of a 1-butyl-3-methylimidazolium cation and a 2,2,3,3,4,4,5,5-octafluoropentanesulfate anion, was washed with a hexane-ethyl acetate (4:1) mixture.

Next, 5 mL of the washed ionic liquid was mixed in 10 mL of acetone, and the resulting acetone solution was poured into a cylindrical dropping funnel (20 mL) in which neutral activated alumina (type I) was packed for about 5 cm. The solution was passed through the funnel under pressurization with an air pump with acetone as a washing solution and then further washed with acetone three times. The resulting acetone solution was concentrated on an evaporator, and the solvent was completely removed by reducing the pressure to 2 Torr. The halide ion impurity content in the ionic liquid purified as above measured by ion chromatography (analytic system: DX-500 (GP40 and ED40) produced by Dionex, column: IonPac AG12A, and AS12A (4 mm (dia)×250 mm), eluent: 0.3 mM NaHCO₃+2.7 mM Na₂CO₃, eluent flow rate: 1.2 mL/min, sample injection: 25 μL, detector: conductometric detection) was 41 ppm.

The purity was measured by reverse phase high performance liquid chromatography (HPLC) at a pH 3 using M-600 and SIC-480 produced by WATERS and equipped with a UV 210 nm JASCO UV-975 detector and a WAKOSIL 5C18 column using 10 mM sodium octanesulfonate as an ion-pairing reagent. The purity was 96.3%.

The purified ionic liquid exhibited hydrophobicity.

### Comparative Example 1

An ionic liquid, 1-butyl-3-methylimidazolium amyl sulfate was synthesized as in EXAMPLE 1 but with ammonium amyl sulfate instead of ammonium 2,2,3,3,4,4,5,5-octafluoropentanesulfate. At room temperature (25° C.), 10 cm³ of the ionic solution obtained was added to 10 cm³ of ion-exchange water placed in a 30 cm³ sample bottle. The bottle was capped, shaken for 5 minutes at a shaking pitch of 30 cm and 60 reciprocating motions per minute, and left to stand for 12 hours. As shown in FIG. 1(b), the ionic liquid was completely mixed with ion-exchange water, thereby forming a liquid with only one phase. The ionic liquid exhibited hydrophilicity. Without purification with neutral activated alumina, the halide ion impurity concentration in the ionic liquid was 155 ppm. The same enzymatic reaction as in EXAMPLE 1 was conducted using the unpurified ionic liquid. The relative rate was as low as 2.9% conv/hr. The results are shown in Table 2.

TABLE 2

| | | | | | | Enzymatic |
|---|---|---|---|---|---|---|
| | | | | Halide | Ionic | reaction |
| | | Purification/ | Hydrophilic/ | ion | conductivity | relative rate |
| | Ionic liquid | washing | hydrophobic | (ppm) | (S/cm) | (% conv/hr) |
| EXAMPLE 7 | 1-Butyl-3- | H₂O | Hydrophobic | 124 | 0.9 × 10⁻³ | 4.2 |
| | methylimidazolium=2,2,3,3,4,4,5,5- | | | | | |
| | octafluoropentanesulfate | | | | | |
| EXAMPLE 8 | 1-Butyl-3- | Saturated | Hydrophobic | 98 | 2.1 × 10⁻³ | 4.2 |
| | methylimidazolium=2,2,3,3,4,4,5,5- | NaHCO₃ + | | | | |
| | octafluoropentanesulfate | H₂O | | | | |
| EXAMPLE 9 | 1-Butyl-3- | MgO/SiO₂ | Hydrophobic | 88 | 2.2 × 10⁻³ | 5.5 |
| | methylimidazolium=2,2,3,3,4,4,5,5- | | | | | |
| | octafluoropentanesulfate | | | | | |
| EXAMPLE 10 | 1-Butyl-3- | Silica gel | Hydrophobic | 98 | 2.0 × 10⁻³ | 3.1 |
| | methylimidazolium=2,2,3,3,4,4,5,5- | | | | | |
| | octafluoropentanesulfate | | | | | |
| COMPARATIVE | 1-Butyl-3- | None | Hydrophilic | 155 | — | 2.9 |
| EXAMPLE 1 | methylimidazolium=amyl sulfate | | | | | |

As is evident from Tables 1 and 2, an ionic liquid containing at least one anion selected from the group consisting of a fluoroalkylsulfate anion, a fluorocycloalkylsulfate anion, and a fluorobenzylsulfate anion exhibits hydrophobicity causing phase separation from water and is widely applicable as electrical device materials, various reaction solvents, etc.

As shown by EXAMPLES 1 to 6 in Table 1, the relative rate of the enzymatic reaction using an ionic liquid having a halide ion concentration of 70 ppm or less is large. As shown by EXAMPLES 1, 2, and 6 in Table 1, the relative rate of the enzymatic reaction using an ionic liquid having a halide ion concentration of 45 ppm or less is large. These facts show that an ionic liquid having a halide ion concentration of 70 ppm or less causes a smaller decrease in enzymatic activity during the enzymatic reaction and is thus preferable as a solvent for the enzymatic reaction.

The embodiments and examples disclosed herein are for illustrative purposes only are thus not limiting. The scope of the present invention is indicated by the claims, not the description above, and includes meanings equivalent to the claims and all modifications within the scope.

### Example 12

An ionic liquid (5 mL), N-butyl-N′-methylimidazolium hexafluorophosphate ([bmim]PF₆) composed of an N-butyl-N′-methylimidazolium cation and a hexafluorophosphate anion was washed with a hexane-ethyl acetate (4:1) mixture.

Next, 5 mL of the washed ionic liquid ([bmim]PF₆) was mixed with 10 mL of acetone, and the resulting acetone solution was poured into a cylindrical dropping funnel (20 mL) in which neutral activated alumina (type I) was packed for about 5 cm. The solution was passed through the funnel under pressurization with an air pump with acetone as a washing solution and then further washed with acetone three times. The resulting acetone solution was concentrated on an evaporator, and the solvent was completely removed by reducing the pressure to 2 Torr. The halide ion impurity content in the ionic liquid [bmim]PF₆ purified as above measured by ion chromatography (analytic system: DX-500 (GP40 and ED40) produced by Dionex, column: IonPac AG12A, and AS12A (4 mm (dia)×250 mm), eluent: 0.3 mM NaHCO₃+2.7 mM Na₂CO₃, eluent flow rate: 1.2 mL/min, sample injection: 25 μL, detector: conductometric detection) was 43 ppm.

The purity was measured by reverse phase high performance liquid chromatography (HPLC) at pH 3 using M-600 and SIC-480 produced by WATERS and equipped with a UV 210 nm JASCO UV-975 detector and a WAKOSIL 5C18 column using 10 mM sodium octanesulfonate as an ion-pairing reagent. The purity was 98.5%.

Furthermore, 1.5 mL of the ionic liquid [bmim]PF₆ purified as above was sampled and mixed with 25 mL of Novozym 435, racemic 5-phenyl-1-penten-3-ol ((±)-1) (49.0 mg, 0.30 mmol), and vinyl acetate (39.0 mg, 0.45 mmol). The resulting mixture was stirred at 35° C. After two hours, 2 mL of diethyl ether was added to separate the liquid into two phases, and the procedure of isolating the ether layer with a pipette was conducted 20 times. The collected ether layer was concentrated under vacuum and purified by thin-layer chromatography on silica gel to isolate a product, i.e., acetate (S)-2, and unreacted 5-phenyl-1-penten-3-ol (R)-1. The reaction scheme therefor is shown in FIG. 1. The optical purities (%ee) of the resulting acetate (S)-2 and 5-phenyl-1-penten-3-ol (R)-1 were measured, and conversions c (% conversion) were calculated from the results. The conversion c (% conversion) calculated was divided by the reaction time (time) to define the relative rate, and the enzymatic activity was evaluated based on the value of the relative rate. The experimental value of the relative rate was 16% conversion/time(h) in this example.

As described below, an ionic liquid [bmim]PF₆ not purified with neutral activated alumina has a high halide ion impurity content. Moreover, the relative rate (16% conversion/time(h)) obtained here is higher than that of the examples in which enzymatic reaction by Novozym 435 is conducted in an ionic liquid [bmim]PF₆ not purified by neutral activated alumina. In other words, purification with neutral activated alumina is extremely effective for removing halide ion impurities in the ionic liquid [bmim]PF₆.

The conductivity of the purified ionic liquid [bmim]PF₆ was 2×10⁻² S/cm. The purified ionic liquid exhibited hydrophobicity.

### Example 13

As in EXAMPLE 12, the relative rate of the enzymatic reaction of [bmim][PF₆] having a purity of 96% was 11% conversion/time(h). The impurity ion concentration was 67 ppm. The conductivity was 9.5×10⁻² S/cm.

### Comparative Example 2

In EXAMPLE 12, the ionic liquid [bmim]PF₆ was not purified with the neutral activated alumina (type I). The halide ion impurity concentration of the ionic liquid [bmim]PF₆ was 130 ppm, and the liquid chromatographic purity was 94.5%. The same enzymatic reaction was also conducted using the ionic liquid [bmim]PF₆ and Novozym 435. The relative rate was 2% conversion/time (h), which was much smaller than that of REFERENCE EXAMPLE 1.

The conductivity of the unpurified ionic liquid [bmim]PF₆ was 6×10⁻³ S/cm.

In other words, the ionic liquid [bmim]PF₆ is not sufficiently purified for causing the above-described enzymatic reaction. Moreover, since the conductivity is lower than that of the purified ionic liquid, optimum performance cannot be expected when the ionic liquid is used for electrochemical devices.

### INDUSTRIAL APPLICABILITY

As described above, the ionic liquid of the present invention exhibits hydrophobicity that causes phase separation from water and can be widely used in various electrical device materials and various reaction solvents.

1. An ionic liquid exhibiting hydrophobicity causing phase separation from water, the ionic liquid comprising at least one anion selected from the group consisting of a fluoroalkylsulfate anion, a fluorocycloalkylsulfate anion, and a fluorobenzylsulfate anion.

2. The ionic liquid according to claim 1, comprising at least one cation selected from the group consisting of a cation having a heterocyclic skeleton containing at least one nitrogen atom and an ammonium cation.

3. The ionic liquid according to claim 1, wherein the halide ion concentration is 70 ppm or less.

4. The ionic liquid according to claim 1, wherein the halide ion concentration is 45 ppm or less.

5. A process for producing an ionic liquid, comprising:
a synthetic step of reacting at least one ammonium salt selected from the group consisting of ammonium fluoroalkylsulfate, ammonium fluorocycloalkylsulfate, and ammonium fluorobenzylsulfate with at least one halide selected from the group consisting of a halide having a heterocyclic skeleton containing at least one nitrogen atom and an ammonium halide, and removing resulting ammonium halide to prepare an ionic liquid; and
a purification step of bringing the ionic liquid into contact with alumina powder to remove halide ions contained in the ionic liquid.

6. The process for producing the ionic liquid according to claim 5, wherein, in the purification step, column chromatography with a column packed with the alumina powder is employed to remove the halide ions contained in the ionic liquid.

7. The process for producing the ionic liquid according to claim 5, wherein the alumina powder is neutral alumina powder.
